(19) United States
(12) Patent Application Publication
(10) Pub. No.: US 2022/0039659 A1
(43) Pub. Date: Feb. 10, 2022

(54) METHOD AND SYSTEM FOR ORAL HYGIENE INSPECTION

(71) Applicant: EMPIDENT GMBH, München (DE)

(72) Inventors: Roland TEVES, München (DE); Alexander KNOCH, Unterföhring (DE)

(73) Assignee: EMPIDENT GMBH, München (DE)

(21) Appl. No.: 17/416,486

(22) PCT Filed: Dec. 6, 2019
(86) PCT No.: PCT/EP2019/084070

§ 371 Date: Jun. 19, 2021

(30) Foreign Application Priority Data
Dec. 20, 2018 (EP) 18214550.8

(51) Int. Cl. A61B 5/00; A61B 1/247; A61B 1/06; A61B 1/00; A61B 1/04

(52) U.S. Cl. CPC A61B 5/0088; A61B 1/247; A61B 1/0623; A61B 1/043; A61B 5/0071; A61B 1/00034; A61B 1/0653

(57) ABSTRACT

A kit configured to perform oral cavity inspection comprising at least one of: an observation component, a light source component and a dental disclosing agent applied on dental surfaces wherein interaction between a line of sight of the observation component and a light beam of the light source component at the dental disclosing agent exhibits an angle of illumination a equal to or less than 35 degrees.

### FIELD

The invention lies in the field of medical technology, such as mirror devices and particularly in the field of mouth mirror devices for oral hygiene inspection.

### INTRODUCTION

Bacteria are ubiquitous microorganisms that can proliferate on diverse surfaces, which also may also be part of other forms. For instance, it is possible to find bacteria on humans such as on the epidermis or on the teeth. A distinctive characteristic of bacteria is their ability to form biofilms, i.e. a mass of bacteria, which may grow, for example, within the mouth of humans. The biofilm allocated on humans' teeth is also known as dental plaque. In general terms, a dental plaque forms on an initial stage a sticky colorless deposit, which may also be considered as an invisible coating onto the teeth. Progressively, the growth of the dental plaque will develop into a brown or pale-yellow layer frequently known as tartar, which further cause oral diseases such as tooth decay, damaging of the tissues of the tooth as result of the bacterial metabolism, and periodontal diseases such as gingivitis.

For this reason, several medical treatment methods have been developed, also known as dental therapy. A frequent approach in dental therapy is the distinction between clean tooth surfaces from surfaces partially or entirely covered by dental plaque, which is frequently achieve, for example, by means of dyes.

Dyes are coloring substances capable of binding to the surface of a substrate. Most used dyes are applied in aqueous solutions, and subsequently required or not a dye fixative, commonly known as mordant. The color of dyes is the result of their chromophore properties, which allow them to absorb defined wavelengths of light. Thus, there are different types of dyes based on the absorbance properties. For instance, there are fluorescent dyes also known as fluorophores, which represent a large group of fluorescent chemical compounds capable of re-emitting light upon light excitation, i.e. the fluorescent dyes become luminescent if excited via a suitable lamp. This type of dyes is frequently used in dental therapy as they are able to adhere to dental plaque, but not to the clean tooth surfaces. Therefore, it is possible to differentiate, via illumination of the teeth, clean dental surfaces from unclean surfaces since dyes do not adhere to clean dental surface, which means that clean dental surfaces can be distinguished as non-fluorescent regions. In simple words, the dental plaque is uncovered and located via illumination of the teeth, opening the possibility for an easy-to-use diagnostic tool to assess oral cleanliness. This technology provides the opportunity to deliver a tool for oral inspection to the public interested in oral hygiene. Hence, several devices have been proposed to provide oral hygiene and oral health checks using fluorescent dyes.

U.S. Pat. No. 3,711,700 A describes an invention for dental, medical and other uses, with a disclosant phosphor dye such as sodium fluorescein, a disclosing light emitting radiation substantially confined to the wavelength range between 380 and 505 nanometers (nm) and free from infrared, yellow and ultraviolet. The light includes a source of light, which can be an incandescent lamp, a dichroic reflector behind it to reflect blue light forwardly and transmit the rest backwardly, a dichroic filter in front of the lamp to transmit blue light and reflect the infrared backwardly, and a viewing mirror for the parts irradiated, the mirror reflecting yellow light toward the observer and transmitting the other radiations. The ultraviolet, although it would excite the fluorescein, is filtered out because it would also make the natural teeth, and some kinds of artificial teeth, fluoresce and obscure the fluorescence of the fluorescein.

Another commercially available device is Gum PlakCheck, which comprises of a dental mirror, containing a source of light for the use of exciting sodium fluorescein. The source of light is a blue light-emitting diode (LED), which is placed in the handle of the dental mirror, the light beam directed to the mirror glass. While checking the teeth using the dental mirror, the teeth surfaces are illuminated by the blue light, photo-exciting the fluorescein previously applied on the dental surface. As the dental mirror is put into the mouth, most dental surfaces can be checked for the presence of dental plaque.

However, these devices do not provide a complete oral hygiene check, as misapprehensions might occur. Some tooth surfaces cannot be inspected, tooth surfaces containing Plaque are hard to see or might be mistaken to be clean even though they are not.

The invention described in U.S. Pat. No. 3,711,700 A is used outside of the mouth, the user looks via a viewing mirror into the mouth and as consequence most tooth surfaces of the upper jaw cannot be inspected, which is a serious limitation for the purpose of oral inspection. Using Gum Plack Check, some of these limitations are overcome as the observer can check most dental surfaces, however there are still some deficiencies to solve other limiting aspects.

In light of the above, it is therefore an object of the present invention to overcome or at least to alleviated the shortcomings and disadvantages of the prior art. More particularly, it is an object of the present invention to provide an easy-to-use and safe diagnostic tool for a complete and accurate inspection of oral hygiene.

### SUMMARY

These objects are met by the present invention.

In a first embodiment, the present invention relates to an observation component configured for inspection of dental surfaces.

The observation may comprise a surface configured to capture fluorescence signals from dental surfaces via at least one observation component, and/or reflect the captured fluorescence signals from dental surfaces.

The observation component may comprise at least one reflecting surface, wherein the reflecting surface may be a mirror.

In one embodiment, the mirror may be a dental mirror. Furthermore, the dental mirror may be a circular mirror with a radius less than 4 cm, more preferably less than 3 cm, most preferably less than 2 cm.

The dental mirror may be a glass mirror.

The dental mirror may be an acrylic mirror.

The at least one dental mirror may be coated with at least one layer of one of aluminum, silver, gold, tin, chrome, silicon oxides, silicon nitrides, etc.

The at least one reflecting surface may be a dichroic filter.

The reflecting surface may be configured to direct the reflected light to a secondary device component.

The reflecting surface may be configured to direct a light beam from a secondary device component.

The observation component may comprise at least one dental camera configured to capture at least one oral cavity image data.

The at least one dental camera may further comprise at least one charge-couple device (CCD).

The at least one dental camera may be configured to transfer the at least one oral cavity image data to a remote device.

The remote device may further comprise at least one display configured to show the at least one image data to a user.

The at least one observation component may further comprise at least one light source.

The observation component may further comprise a body configured to hold the observation component.

The remote device may be an electronic display, such as, for example, a mobile phone, a monitor, a tablet, etc.

The dichroic filter may be configured to direct excitation light of at least one light source to dental surfaces.

The body may comprise an elongated holding handle, which may be made of corrosion and/or temperature resistance material comprising one of: stainless steel, titanium alloys, zirconium alloys, molybdenum alloys, tantalum alloys, copper alloys such as bronze and/or brass, graphite, aluminosilicates, pure silica, quartz glass, andesite and/or basalt coated materials, polyfluoroethylene resins, polyethylene, polystyrene, etc.

The observation component may collect light from a field of view larger than 1 steradian, more preferably larger than 2 steradian, most preferably larger than 3 steradian.

The observation component may be configured to collect light from a plurality of directions from any points within the field of view.

The collection of light approximately may exhibit a spherical symmetry with respect to the center of the observation component.

In a further embodiment, the present invention relates to a light source component configured for inspection of dental surfaces.

The light source component may comprise at least one light source.

The light source may be placed in the elongated holding handle.

The light source component may comprise of at least one light source configured to provide a beam angle larger than 30 degrees, more preferably larger than 60 degrees, most preferably larger than 90 degrees.

The emission of light approximately may exhibit a spherical symmetry with respect to the center of the observation component.

The at least one light source component may further comprise a plurality of light sources placed on the periphery of the observation component.

The at least one light source may be placed behind at least one dichroic filter.

The light source component may further comprise different light sources of different wavelengths.

The light source component may further comprise a plurality of positions on the periphery of the observation component.

The light source component may comprise of at least one light source, placed at a distance of less than 10 cm from the center of the observation component, more preferably less than 6 cm, most preferably less than 4 cm.

The light source component may comprise of a reflecting surface, illuminated by a focused beam of light wherein the reflecting surface may be placed at a distance of less than 10 cm from the center of the observation component, more preferably less than 6 cm, most preferably less than 4 cm.

The light source may comprise a distal end of an optical fiber wherein the distal end may be placed at a distance of less than 10 cm from the center of the observation component, more preferably less than 6 cm, most preferably less than 4 cm.

The at least one light source may be placed on a secondary device component.

The light source component may be configured to provide an illumination beam directed on the observation component.

The light source component may be configured to provide a difference of an angle of reflection between the illumination beam and a line of sight on the observation component of less than 35 degrees, more preferably less than 25 degrees, most preferably less than 15 degrees.

The light source component may be configured to emit light in a wavelength range between 200 and 750 nm, more preferably between 250 and 700 nm, most preferably between 300 and 600 nm.

The light source component may further comprise at least one optical filter.

The at least one light source may further comprise at least one electronic control mechanism configured to switch on and off the at least one light source component.

The at least one electronic control mechanism may further comprise tuning parameters of the at least one light source, such as, for example, intensity, wavelength, etc.

The light source may be used to excite a dental disclosing agent.

The at least one light source configured to provide a point-source illumination. The point-source illumination may be emitted approximately as a radial illumination from the center of the observation component.

In a further embodiment, the present invention also relates to a filtering component configured for inspection of dental surfaces. The filtering component may be configured to increase the contrast of the fluorescent signal of a dental disclosing agent.

The filtering component may be configured to decrease the intensity of light not exhibiting wavelengths of the fluorescent signal.

The filtering component may further comprise at least one optical filter.

The at least one optical filter may further comprise at least one optical long pass filter.

The at least one optical filter may be placed in a plurality of different positions within the light of sight.

The filtering component may be placed on the observation component.

The filtering component may be placed on the elongated holding handle.

The filtering component may be placed on a secondary device component.

The filtering component may be placed on an external mirror.

The filtering component may be placed in front of a camera lens.

The filtering component may comprise of a spectral sensitivity of a camera photosensor, i.e. a spectral sensitive sensor.

The filtering component may comprise of a filtering software processing component.

The filtering component may be configured to pass on wavelengths in a range between 350 and 700 nm, more preferably between 400 and 650 nm, most preferably between 450 and 600 nm.

The filtering component may be a longpass filter, exhibiting a cut-off wavelength in a range of ±50 nm, more preferably ±40 nm, most preferably ±20 nm of the emission wavelength of a dental disclosing agent.

The filtering component may further be configured to achieve a Michelson contrast between the dental plaque disclosing agent on dental surfaces and the clean dental surface higher than 0.03, more preferably higher than 0.04, most preferably higher than 0.05.

In a further embodiment, the present invention relates to a dental disclosing agent configured for disclosing the presence of dental plaque. The dental disclosing agent may comprise at least one dye, for example, but not limited to, iodiones (such as iodine crystals, potassium iodide, zinc iodide), azo dyes (e.g. bismarck brown Y), merbromin, erythrosine, fast green, fluorescein, fuchsin, riboflavin, etc., and their derivatives.

The dye may comprise at least one water soluble compound.

The dye may be configured to exhibit fluorescence emission wavelengths in a range between 200 and 700 nm, more preferably between 250 and 650 nm, most preferably between 300 and 600 nm.

The dental disclosing agent embodiments wherein the dye may further comprise at least one of:
High quantum yield (0.8-1);
Biocompatibility;
Small molecules/high diffusion rate; and
Plaque-binding properties.

The disclosing agent may be a liquid, such as, for example, a ready to use solution, a concentrated solution, etc.

The disclosing agent may be a solid, such as, for example, tablets.

In a further embodiment, the present invention also relates to an energy source component configured to provide energy to a plurality of components for oral cavity inspection. The energy source component may comprise at least one energy source.

The at least one energy source may comprise at least one battery.

The at least one battery may be a rechargeable battery and/or accumulator.

The at least one battery may be a replaceable battery and/or replaceable accumulator.

The at least one energy source may be at least one of: a transformer and a capacitor.

The energy source component may comprise at least one electronic control mechanism configured to switch on and off the at least one energy source.

In a further embodiment, the present invention relates to a kit configured to perform oral cavity inspection. The kit may comprise at least one of: an observation component, a light source component, and a disclosing agent component, wherein the interaction between a line of sight of the observation component and a light beam of the light source component with the disclosing agent component exhibits an angle of illumination a equal to or less than 35 degrees.

The angle of illumination a may be less than 25 degrees, more preferably less than 15 degrees.

The kit may further comprise a filtering component.

The filtering component may further comprise at least one of: an optical filter, a spectral sensitive sensor and a filtering software.

The filtering component may be configured for passing the fluorescent emission of the dental disclosing agent and blocking light of wavelengths via selectively filtering light in a wavelength range between 350 and 700 nm, more preferably between 400 and 650 nm, most preferably between 450 and 600 nm.

The kit may further comprise an energy source component.

The kit may further a comprise secondary device comprising at least one of: an observation component, a filtering component, a light source component, and an energy source component.

The light source component may be configured to illuminate more than 30%, more preferably more than 50%, most preferably more than 70% of the field of view of the observation component.

The light source component may further be configured to illuminate more than 30%, more preferably more than 50%, most preferably more than 70% of the illuminated field of view of the observation component with an angle of illumination a equal to or less than 35 degrees, more preferably less than 25 degrees, most preferably less than 15 degrees.

The light source component and the center of the observation component may be placed at a distance of less than 10, more preferably less than 6 cm, most preferably less than 4 cm.

The kit may further comprise a secondary device component.

The secondary device component may comprise at least one of: a second observation component, a filtering component, a light source component, and an energy source component.

The second observation component may comprise at least one of: a remote observation device, an external camera, and an external mirror.

The secondary device component may be made of corrosion and/or temperature resistance material comprising one of: stainless steel, titanium alloys, zirconium alloys, molybdenum alloys, tantalum alloys, copper alloys such as bronze and/or brass, graphite, aluminosilicates, pure silica, quartz glass, andesite and/or basalt coated materials, polyfluoroethylene resins, polyethylene, polystyrene, etc.

The secondary device component may further be configured to provide a light beam directed to the observation component.

The light beam of the light source component and a line of sight of the observation component may exhibit a difference of a reflection angle less than 35 degrees, more preferably less than 25 degrees, most preferably less than 15 degrees.

The light source component of the secondary device may further be configured to illuminate more than 30%, more preferably more than 50%, most preferably more than 70% of the illuminated field of view of the observation component with an angle of illumination a equal to or less than 35 degrees, more preferably less than 25 degrees, most preferably less than 15 degrees.

The light source component of the secondary device component may be configured to place the light source component at a distance of less than 15 cm to the line of sight, more preferably less than 10 cm, most preferably less than 6 cm, and may further comprise at least one electronic control mechanism configured to switch on and off the light source component.

The secondary device component may comprise a mechanism to be fixed on at least one external mirror surface.

The at least one external mirror may be a parabolic mirror configured to magnify the image of the observation component at least 2×, more preferably at least 4×, most preferably at least 6× the image size.

The light source may be placed in the center of the parabolic mirror.

The secondary device component may comprise a headband comprising at least one light source.

The secondary device component may comprise glasses with at least one light source.

The secondary device component may comprise a camera.

The light source component of secondary device component may be placed at a distance of less than 15 cm to the second observation component, more preferably less than 10 cm, most preferably less than 6 cm.

The secondary device component may comprise a mechanism to fix the secondary device component on camera device comprising a camera, such as, for example, a smartphone.

The secondary device may further comprise a filtering component according to any of the filtering component embodiments.

The filtering component may be configured to achieve a Michelson contrast higher than 0.03, more preferably higher than 0.04, most preferably higher than 0.05.

Furthermore, the present invention also relates to a method comprising using any of the above described features to perform oral cavity inspection.

The present invention also relates to the use of the method for using any of the above described features to perform oral cavity inspection.

The term observation component may be intended to describe a surface capable of collecting light, which may be for example a reflecting surface. The observation component may also be a surface configured for collecting light. The collection of light may be conceptually described via the field of view (FoV). The FoV of the observation component may be modeled as a surface on a geometrical sphere. Furthermore, the field of view may be parameterized via a solid angle Ω, whereas the apex of the solid angle Ω may be the center point of the observation component. The term solid angle Ω, or the term field of view (FoV) may also be intended to define the solid angle through which the observation component, for instance, a mirror and/or a detector, may be sensitive to electromagnetic radiation.

The term light source component may be intended to describe a component which may be configured to emit light of a given range of wavelengths, which may further exhibit a defined geometry.

Furthermore, the observation component and the light source component may be configured to illuminate all points P on any surface within the field of view, further exhibiting an illumination angle α most preferably lower than 15 degrees.

In approximation, the field of view may exhibit a spherical symmetry. Both components (observation component and light source component) may be configured to provide an illumination angle α smaller than 35 degrees (for all Points P within the illuminated field of view). In other words, while interacting with the dental disclosing agent, the light source component may need to exhibit the same spherical symmetry as the observation component. This defined geometry of the light source component may also be referred to as point-source illumination, as it may be the symmetry comparable to a mathematical modeled point-source of light, placed in the apex in the center of the observation component (at Point G).

The present technology is also defined by the following numbered embodiments.

Below, observation component embodiments will be discussed. These embodiments are abbreviated by the letter “O” followed by a number. When reference is herein made to a system embodiment, those embodiments are meant.

O1. An observation component (102) configured for inspection of dental surfaces.

O2. The observation component (102) according to the preceding embodiment wherein the observation component (102) comprises a surface configured to
capture fluorescence signals from dental surfaces via at least one observation component; and/or
reflect the captured fluorescence signals from dental surfaces.

O3. The observation component (102) according to the preceding embodiment wherein the observation component (102) comprises at least one reflecting surface.

O4. The observation component (102) according to the preceding embodiment wherein the reflecting surface is a mirror.

O5. The observation component (102) according to the preceding embodiment wherein the mirror is a dental mirror.

O6. The observation component (102) according to the preceding embodiment wherein the dental mirror is a circular mirror with a radius less than 4 cm, more preferably less than 3 cm, most preferably less than 2 cm.

O7. The observation component (102) according to any of the preceding embodiments wherein the dental mirror is a glass mirror.

O8. The observation component (102) according to any of the embodiments O1 to O6 wherein the dental mirror is an acrylic mirror.

O9. The observation component (102) according to any of the preceding embodiments wherein the at least one dental mirror is coated with at least one layer of one of aluminum, silver, gold, tin, chrome, silicon oxides, silicon nitrides, etc.

O10. The observation component (102) according to embodiment O3 wherein the at least one reflecting surface is a dichroic filter.

O11. The observation component (102) according to any of the preceding embodiments O3 to O10 wherein the reflecting surface is configured to direct the reflected light to a secondary device component.

O12. The observation component (102) according to any of the preceding embodiments wherein the reflecting surface is configured to direct a light beam from a secondary device component.

O13. The observation component (102) according to embodiment O2 wherein the observation component (102) comprises at least one dental camera configured to capture at least one oral cavity image data.

O14. The observation component (102) according to the preceding embodiment wherein the at least one dental camera further comprises at least one charge-couple device (CCD).

O15. The observation component (102) according to the preceding embodiment wherein the at least one dental camera is configured to transfer the at least one oral cavity image data to a remote device.

O16. The observation component (102) according to the preceding embodiment wherein the remote device further comprises at least one display configured to show the at least one image data to a user.

O17. The observation component (102) according to any of the preceding embodiments wherein the at least one observation component further comprises at least one light source.

O18. The observation component (102) according to any of the preceding embodiments wherein the observation component (102) further comprises a body configured to hold the observation component (102).

O19. The observation component (102) according to embodiment O15 wherein the remote device is an electronic display, such as, for example, a mobile phone, a monitor, a tablet, etc.

O20. The observation component (102) according to preceding embodiment wherein the dichroic filter is configured to direct excitation light of at least one light source to dental surfaces.

O21. The observation component (102) according to any of the preceding embodiments and with features of O18, wherein the body comprises an elongated holding handle.

O22. The observation component (102) according to any of the preceding embodiments wherein the elongated holding handle is made of corrosion and/or temperature resistance material comprising one of: stainless steel, titanium alloys, zirconium alloys, molybdenum alloys, tantalum alloys, copper alloys such as bronze and/or brass, graphite, aluminosilicates, pure silica, quartz glass, andesite and/or basalt coated materials, polyfluoroethylene resins, polyethylene, polystyrene, etc.

O23. The observation component (102) according to any of the preceding embodiments wherein the observation component (102) collects light from a field of view larger than 1 steradian, more preferably larger than 2 steradian, most preferably larger than 3 steradian.

O24. The observation component (102) according to any of the preceding embodiments wherein the observation component (102) is configured to collect light from a plurality of directions from any points within the field of view.

O25. The observation component (102) according to any of the preceding embodiments wherein the collection of light approximately exhibits a spherical symmetry with respect to the center of the observation component (102).

Below, light source component embodiments will be discussed. These embodiments are abbreviated by the letter “L” followed by a number. When reference is herein made to a system embodiment, those embodiments are meant.

L1. A light source component (104) configured for inspection of dental surfaces.

L2. The light source component (104) according to the preceding embodiment further comprising at least one light source.

L3. The light source component (104) according to any of the preceding embodiments and with features of embodiment O21 wherein light source is placed in the elongated holding handle.

L4. The light source component (104) according to the preceding embodiment wherein the light source component (104) comprises of at least one light source configured to provide a beam angle larger than 30 degrees, more preferably larger than 60 degrees, most preferably larger than 90 degrees.

L5. The light source component (104) according to the preceding embodiment wherein the emission of light approximately exhibits a spherical symmetry with respect to the center of the observation component (102).

L6. The light source component (104) according to the preceding embodiment wherein the at least one light source component further comprises a plurality of light sources placed on the periphery of the observation component (102).

L7. The light source component (104) according the preceding embodiment wherein the at least one light source is placed behind at least one dichroic filter.

L8. The light source component (104) according to any of the preceding light source component embodiments wherein the light source component (104) further comprises different light sources of different wavelengths.

L9. The light source component (104) according to L6, wherein the light source component (104) further comprises a plurality of positions on the periphery of the observation component (102).

L10. The light source component (104) according to any of the preceding light source component embodiments wherein the light source component (104) comprises of at least one light source, placed at a distance of less than 10 cm from the center of the observation component (102), more preferably less than 6 cm, most preferably less than 4 cm.

L11. The light source component (104) according to any of the preceding light source component embodiments wherein the light source component (104) comprises of a reflecting surface, illuminated by a focused beam of light wherein the reflecting surface is placed at a distance of less than 10 cm from the center of the observation component (102), more preferably less than 6 cm, most preferably less than 4 cm.

L12. The light source according to any of the preceding light source component embodiments wherein the light source comprises a distal end of an optical fiber wherein the distal end is placed at a distance of less than 10 cm from the center of the observation component (102), more preferably less than 6 cm, most preferably less than 4 cm.

L13. The light source component (104) according to preceding embodiment, wherein the at least one light source is placed on a secondary device component.

L14. The light source component (104) according to preceding embodiment, wherein the light source component (104) is configured to provide an illumination beam directed on the observation component (102).

L15. The light source component (104) according to preceding embodiment, wherein the light source component (104) is configured to provide a difference of an angle of reflection between the illumination beam and a line of sight on the observation component (102) of less than 35 degrees, more preferably less than 25 degrees, most preferably less than 15 degrees.

L16. The light source component (104) according to any of the preceding light source component embodiments wherein the light source component (104) is configured to emit light in a wavelength range between 200 and 750 nm, more preferably between 250 and 700 nm, most preferably between 300 and 600 nm.

L17. The light source component (104) according to L17 wherein the light source component (104) further comprises at least one optical filter.

L18. The light source component (104) according to any of the light source component (104) embodiments wherein the at least one light source further comprises at least one electronic control mechanism configured to switch on and off the at least one light source component.

L19. The light source component (104) according to the preceding embodiment wherein the at least one electronic control mechanism further comprises tuning parameters of the at least one light source, such as, for example, intensity, wavelength, etc.

L20. The light source component (104) according to any of the preceding light source component embodiments and with features of embodiment L16 wherein the light source is used to excite a dental disclosing agent.

L21. The light source component (104) according to any of the preceding light source component embodiments wherein the at least one light source configured to provide a point-source illumination.

L22. The light source component (104) according to any of the preceding light source component embodiments, wherein the point-source illumination is emitted approximately as a radial illumination from the center of the observation component (102).

Below, filtering component embodiments will be discussed. These embodiments are abbreviated by the letter “F” followed by a number. When reference is herein made to a system embodiment, those embodiments are meant.

F1. A filtering component (130) configured for inspection of dental surfaces.

F2. The filtering component (130) according to the preceding embodiment wherein the filtering component (130) is configured to increase the contrast of the fluorescent signal of a dental disclosing agent.

F3. The filtering component (130) according to any of the preceding filtering component embodiments wherein the filtering component (130) is configured to decrease the intensity of light not exhibiting wavelengths of the fluorescent signal.

F4. The filtering component (130) according to the any of the two preceding embodiments further comprising at least one optical filter.

F5. The filtering component (130) according to the preceding embodiment wherein the at least one optical filter further comprises at least one optical long pass filter.

F6. The filtering component (130) according to the preceding embodiment wherein the at least one optical filter in placed in a plurality of different positions within the light of sight.

F7. The filtering component (130) according to the preceding embodiment wherein the filtering component (130) is placed on the observation component (102).

F8. The filtering component (130) according to embodiment F6 and with features of embodiment O21 wherein the filtering component (130) is placed on the elongated holding handle.

F9. The filtering component (130) according to embodiment F6 wherein the filtering component (130) is placed on a secondary device component.

F10. The filtering component (130) according to embodiment F6 wherein the filtering component (130) is placed on an external mirror.

F11. The filtering component (130) according to embodiment F6 wherein the filtering component (130) is placed in front of a camera lens.

F12. The filtering component (130) according to embodiment F2 wherein the filtering component (130) comprises of a spectral sensitivity of a camera photosensor, i.e. a spectral sensitive sensor.

F13. The filtering component (130) according to embodiment F2 wherein the filtering component (130) comprises of a filtering software processing component.

F14. The filtering component (130) according to any of the preceding filtering component embodiments wherein the filtering component (130) is configured to pass on wavelengths in a range between 350 and 700 nm, more preferably between 400 and 650 nm, most preferably between 450 and 600 nm.

F15. The filtering component (130) according component to any of the preceding filtering component embodiments wherein the filtering component (130) is a longpass filter, exhibiting a cut-off wavelength in a range of ±50 nm, more preferably ±40 nm, most preferably ±20 nm of the emission wavelength of a dental disclosing agent.

F16. The filtering component (130) according to any of the preceding filtering component embodiments wherein the filtering component (130) is further configured to achieve a Michelson contrast between the dental plaque disclosing agent on dental surfaces and the clean dental surface higher than 0.03, more preferably higher than 0.04, most preferably higher than 0.05.

Below, dental disclosing agent embodiments will be discussed. These embodiments are abbreviated by the letter “D” followed by a number. When reference is herein made to a system embodiment, those embodiments are meant.

D1. A dental disclosing agent configured for disclosing the presence of dental plaque.

D2. The dental disclosing agent according to the preceding embodiment wherein the dental disclosing agent comprises at least one dye, for example, but not limited to, iodiones (such as iodine crystals, potassium iodide, zinc iodide), azo dyes (e.g. bismarck brown Y), merbromin, erythrosine, fast green, fluorescein, fuchsin, riboflavin, etc., and their derivatives.

D3. The dental disclosing agent according to any of the preceding dental disclosing agent embodiments wherein the dye comprises at least one water soluble compound.

D4. The dental disclosing agent according to any of the preceding dental disclosing agent embodiments wherein the dental disclosing agent is configured to exhibit fluorescence emission wavelengths in a range between 200 and 700 nm, more preferably between 250 and 650 nm, most preferably between 300 and 600 nm.

D5. The dental disclosing agent according to any of the preceding dental disclosing agent embodiments wherein the dental disclosing agent is further configured for absorbing light in a wavelength range between 200 and 650 nm, more preferably between 300 and 600 nm, most preferably between 400 and 550 nm.

D6. The dental disclosing agent according to any of the preceding dental disclosing agent embodiments wherein the dye further comprises at least one of:
High quantum yield (0.8-1);
Biocompatibility;
Small molecules/high diffusion rate; and
Plaque-binding properties.

D7. The dental disclosing agent according to any of the preceding dental disclosing agent embodiments wherein the disclosing agent is a liquid, such as, for example, a ready to use solution, a concentrated solution, etc.

D8. The dental disclosing agent according to any of the preceding dental disclosing agent embodiments D1 to D6 wherein the disclosing agent is a solid, such as, for example, tablets.

Below, energy source component embodiments will be discussed. These embodiments are abbreviated by the letter “E” followed by a number. When reference is herein made to a system embodiment, those embodiments are meant.

E1. An energy source component configured to provide energy to a plurality of components for oral cavity inspection.

E2. The energy source component according to the preceding embodiment comprising at least one energy source.

E3. The energy source component according to the preceding embodiment wherein the at least one energy source comprises at least one battery.

E4. The energy source component according to the preceding embodiments wherein the at least one battery is a rechargeable battery and/or accumulator.

E5. The energy source component according to any of the preceding two embodiments wherein the at least one battery is a replaceable battery and/or replaceable accumulator.

E6. The energy source component according to embodiment E2 wherein the at least one energy source is at least one of
a transformer; and
a capacitor.

E7. The energy source component according to any of the preceding energy source component embodiments further comprising at least one electronic control mechanism configured to switch on and off the at least one energy source.

Below, kit embodiments will be discussed. These embodiments are abbreviated by the letter “K” followed by a number. When reference is herein made to a system embodiment, those embodiments are meant.

K1. A kit (100) configured to perform oral cavity inspection.

K2. The kit (100) according to the preceding embodiment further comprising at least one of
an observation component (102) according to any of the preceding observation component embodiments;
a light source component (104) according to any of the preceding light source component embodiments; and
a disclosing agent component according to any of the preceding disclosing agent components,

wherein the interaction between a line of sight of the observation component (102) and a light beam of the light source component (104) with the disclosing agent component exhibits an angle of illumination (α) equal to or less than 35 degrees.

K3. The kit according to any of the preceding kit embodiments wherein the angle of illumination (α) is less than 25 degrees, more preferably less than 15 degrees.

K4. The kit (100) according to any of the preceding kit embodiments wherein the kit (100) further comprising a filtering component (130) according to any of the preceding filtering component embodiments wherein the filtering component (130) further comprises at least one of
an optical filter;
a spectral sensitive sensor; and
a filtering software,

wherein the filtering component (130) is configured for passing the fluorescent emission of the dental disclosing agent and blocking light of wavelengths via selectively filtering light in a wavelength range between 350 and 700 nm, more preferably between 400 and 650 nm, most preferably between 450 and 600 nm.

K5. The kit (100) according to any of the preceding kit embodiments wherein the kit (100) further comprising an energy source component according to any of the preceding energy source embodiments.

K6. The kit (100) according to any of the preceding kit (100) embodiment wherein the light source component (104) is configured to illuminate more than 30%, more preferably more than 50%, most preferably more than 70% of the field of view of the observation component (102).

K7. The kit (100) according to any of the preceding embodiments wherein the light source component (104) is further configured to illuminate more than 30%, more preferably more than 50%, most preferably more than 70% of the illuminated field of view of the observation component (102) with an angle of illumination (α) equal to or less than 35 degrees, more preferably less than 25 degrees, most preferably less than 15 degrees.

K8. The kit (100) according to the preceding embodiment wherein the light source component (104) and the center of the observation component (102) is placed ata distance of less than 10, more preferably less than 6 cm, most preferably less than 4 cm.

K9. The kit (100) according to any of the preceding embodiments wherein the kit (100) further comprising a secondary device comprising at least one of
an observation component (102) according to any of the preceding observation component embodiments;
a filtering component (130) according to any of the preceding filtering component embodiments;
a light source component (104) according to any of the preceding light source component embodiments; and
   an energy source component according to any of the preceding energy source component embodiments.

K10. The kit (100) according to the preceding embodiment, wherein the second observation component comprises at least one of
a remote observation device;
an external camera; and
an external mirror.

K11. The kit (100) according to any of the preceding two embodiments wherein the secondary device component is made of corrosion and/or temperature resistance material comprising one of: stainless steel, titanium alloys, zirconium alloys, molybdenum alloys, tantalum alloys, copper alloys such as bronze and/or brass, graphite, aluminosilicates, pure silica, quartz glass, andesite and/or basalt coated materials, polyfluoroethylene resins, polyethylene, polystyrene, etc.

K12. The kit (100) according to embodiment K9 wherein the light source component (104) of the secondary device component is further configured to provide a light beam directed to the observation component (102).

K13. The kit (100) according to the preceding embodiment and with features of embodiment K9 wherein light beam of the light source component (104) and a line of sight of the observation component (102) exhibit a difference of a reflection angle less than 35 degrees, more preferably less than 25 degrees, most preferably less than 15 degrees.

K14. The kit (100) according to the preceding embodiment wherein the light source component (104) of the secondary device is further configured to illuminate more than 30%, more preferably more than 50%, most preferably more than 70% of the illuminated field of view of the observation component (102) with an angle of illumination (α) equal to or less than 35 degrees, more preferably less than 25 degrees, most preferably less than 15 degrees.

K15. The kit (100) according to the preceding embodiment wherein the light source component (104) of the secondary device component is configured to place the light source component (104) at a distance of less than 15 cm to the line of sight, more preferably less than 10 cm, most preferably less than 6 cm, and further comprises at least one electronic control mechanism configured to switch on and off the light source component (104).

K16. The kit (100) according to the preceding embodiment wherein the secondary device component comprises a mechanism to be fixed on at least one external mirror surface.

K17. The kit (100) according to the preceding embodiment wherein the at least one external mirror is a parabolic mirror configured to magnify the image of the observation component (102) at least 2×, more preferably at least 4×, most preferably at least 6× the image size.

K18. The kit (100) according to the preceding embodiment wherein the light source is placed in the center of the parabolic mirror.

K19. The kit (100) according to any of the preceding kit embodiments and with feature of embodiment K9 wherein the secondary device component comprises a headband comprising at least one light source.

K20. The kit (100) according to any of the preceding kit embodiments and with feature of embodiment K10 wherein the secondary device component comprises glasses with at least one light source.

K21. The kit (100) according to any of the preceding kit embodiments and with feature of embodiment K9 wherein the secondary device component comprises a camera.

K22. The kit (100) according to the preceding embodiment and with features of embodiment K9 wherein the light source component (104) of secondary device component is placed at a distance of less than 15 cm to the second observation component, more preferably less than 10 cm, most preferably less than 6 cm.

K23. The kit (100) according to the preceding embodiment wherein the secondary device component comprises a mechanism to fix the secondary device component on camera device comprising a camera, such as, for example, a smartphone.

K24. The kit (100) according to any preceding embodiments wherein the secondary device further comprises a filtering component (130) according to any of the filtering component (130) embodiments.

K25. The kit (100) according to the preceding embodiment wherein the filtering component (130) is configured to achieve a Michelson contrast higher than 0.03, more preferably higher than 0.04, most preferably higher than 0.05.

Below, method embodiments will be discussed. These embodiments are abbreviated by the letter “M” followed by a number. When reference is herein made to a use embodiment, those embodiments are meant.

M1. A method comprising using any of the preceding embodiments to perform oral cavity inspection.

Below, use embodiments will be discussed. These embodiments are abbreviated by the letter “U” followed by a number. When reference is herein made to a use embodiment, those embodiments are meant.

U1. Use of the method according to any of the preceding method embodiments in oral cavity inspection.

The present invention will now be described with reference to the accompanying drawings which illustrate embodiments of the invention. These embodiments should only exemplify, but not limit, the present invention.

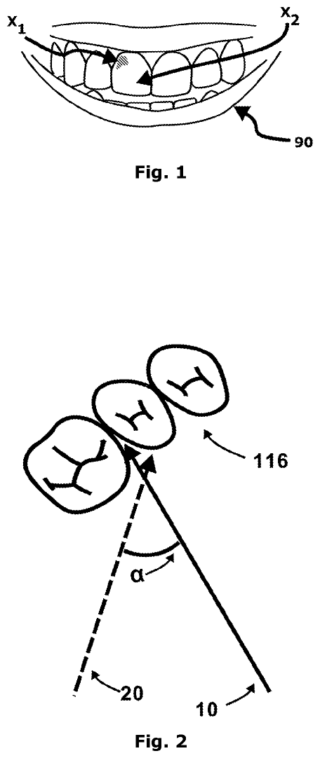

FIG. 1 depicts an oral cavity exhibiting the incisor teeth;

FIG. 2 depicts concepts of illumination of dental surfaces;

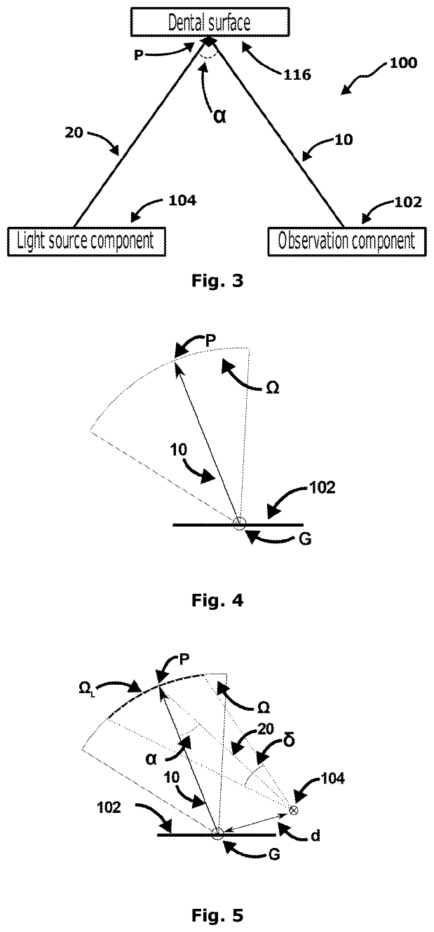

FIG. 3 depicts concepts of illumination of dental surfaces via a kit according to embodiments of the present invention;

FIG. 4 depicts generalized concepts of an observation component according to embodiments of the present invention;

FIG. 5 depicts generalized concepts of interaction between a light source component and an observation component according to embodiments of the present invention;

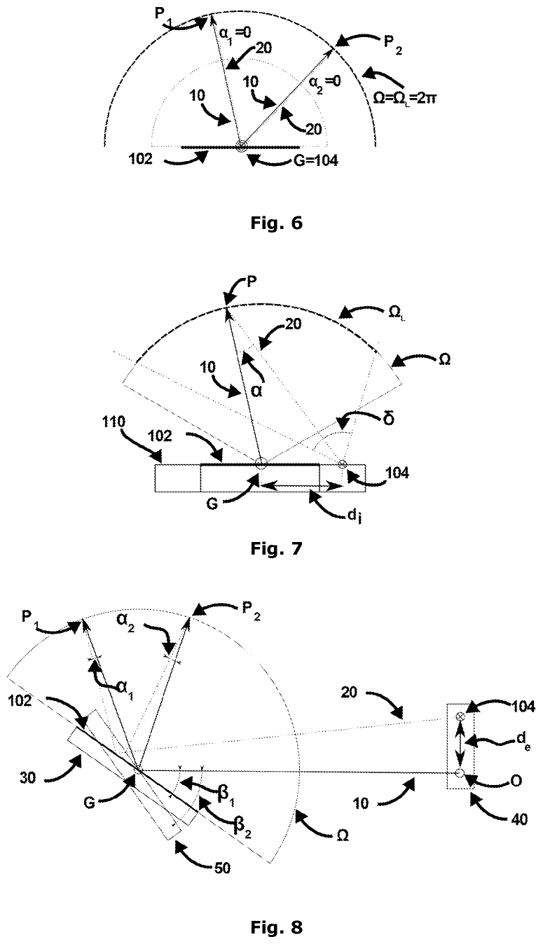

FIG. 6 depicts generalized concepts of concepts of point-source illumination according to embodiments of the present invention;

FIG. 7 depicts a dental device according to static solution embodiments of the present invention;

FIG. 8 depicts a dental device and a secondary device according to dynamic solution embodiments of the present invention;

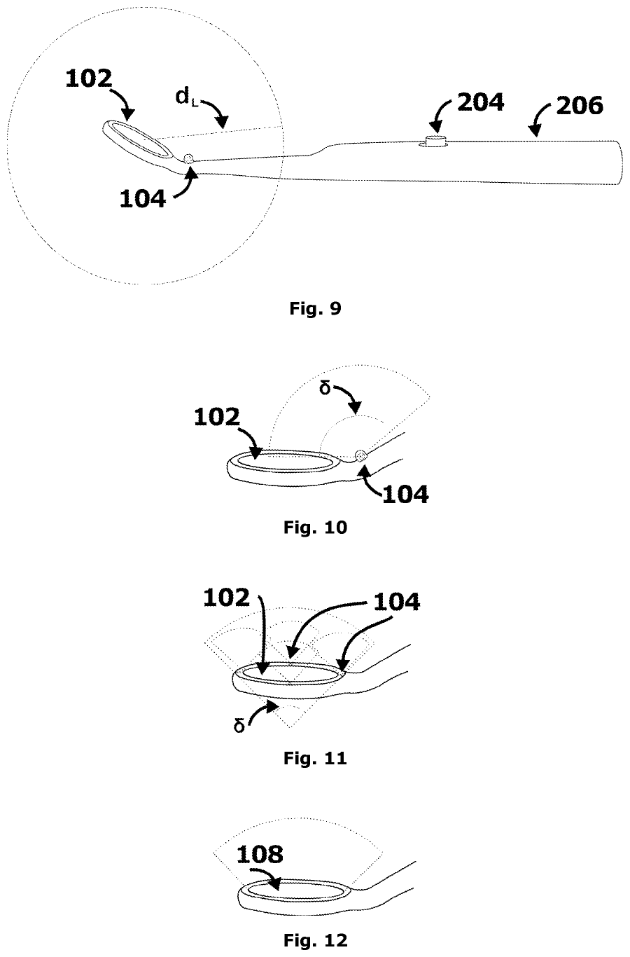

FIG. 9 depicts a dental device for inspection of oral cavities according to embodiments of the present invention;

FIG. 10-15 depict exemplary embodiments for different positioning of the light source component according to embodiments of the present invention;

FIG. 14-16 depict a dental camera as observation component according to embodiments of the present invention;

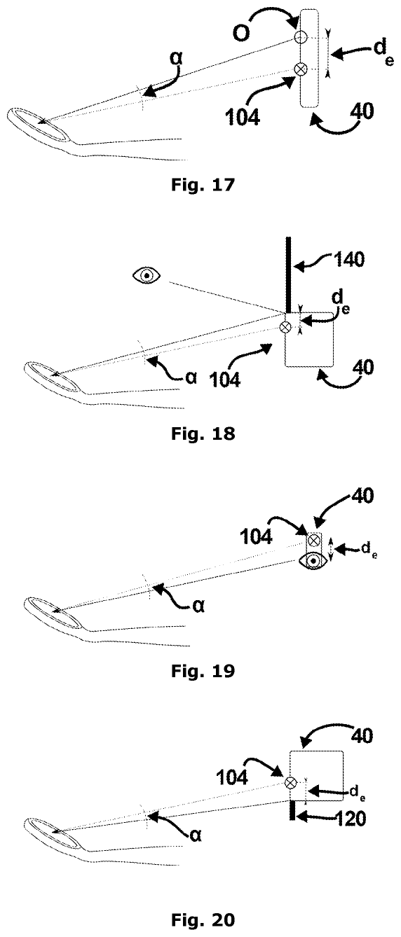

FIG. 17-28 depicts secondary devices for inspection of oral cavities according to embodiments of the present invention;

It is noted that not all the drawings carry all the reference sings. Instead, in some of the drawings, some of the reference sings have been omitted for sake of the brevity and simplicity of illustration. Embodiments of the present invention will now be described with reference to the accompanying drawings.

FIG. 1 schematically depicts an oral cavity exhibiting the incisor teeth conceptually identify by reference numeral 90. On an incisor is observed a schematic representation of dental plaques conceptually identified by reference numeral X1. Further, a cleaned dental surface is conceptually identified by reference numeral X2. Dental plaque X1 is a variable structural that builds up on dental surfaces as result of the sequential colonization and growth or microorganisms in the oral cavity. Such a variable structure build-up may also be referred to as a biofilm deposition, biofilm growth and/or biofilm formation, which after a consistent and significant deposition on dental surfaces may form dental plaques. Dental plaques represent an endangering actor for the dental health, since it may contribute to the deterioration of dental surfaces and other associated gingival health conditions as a result of bacterial metabolic products and substance from saliva and blood.

Frequently the dental plaque X1 is not easily observed with naked eyes until the accumulation of the bacterial biofilm is considerable high and may be able to form an easily observable plaque deposition on a dental surface. However, it may be possible to differentiate between a clean dental surface from a dental surface covered by dental plaque X1 from the early stages of the biofilm formation. Such a differentiation may be possible using different observation tools such as a dental mirror in combination with dental plaque X1 disclosing agent and, when necessary, an adequate light source to illuminate the dental surfaces 116, for instance, in case of a fluorescent disclosing agent.

In case of fluorescent dyes, an excitation light may be needed to photoexcite the fluorescent dye, which may also be diffusely reflected by the dental surface 116, resulting in bad contrast, obscuring the fluorescence of the dyed plaque. In order to differentiate between a clean tooth surface and a tooth surface covered by dental plaque, a difference in contrast may be needed, for example, a difference in Michelson contrast.

Michelson contrast may be defined via the following equation:

Kₘ=I₁−I₂/I₁+I₂ with 0≤Kₘ≤1

where Km is the Michelson contrast, I1 the intensity of the zone X1, which may also be referred to as plaque surface X1 and I2 the intensity of the zone X2, which may also be referred to as clean tooth surface X2. The higher the Michelson contrast may get, the clearer the dental plaque X1 may be observed.

As a fluorescence signal has a different wavelength to that of a diffuse reflection, a filter can be used to lower the intensity depending on the wavelength. Passing the wavelengths of the fluorescence signal and blocking the wavelength of the diffuse reflection, the contrast may be increased, as 12 is getting lower.

In one embodiment of the present invention, a dental mirror may be used as a dental device 100, which may further comprise a filter comprising of an optical longpass filter.

In another embodiment, the dental device 100 may comprise a dental camera 120, and the filter might comprise at least one of an optical longpass filter in front of the dental camera 120, a selective photosensitivity of a photo-chip and/or a software solution.

FIG. 2 schematically depicts concepts of illumination of dental surfaces 116. In simple terms, it comprises a line of sight conceptually identified by reference numeral 10, an illumination beam conceptually identified by reference numeral 20 under an illumination angle conceptually identified by reference numeral α. The illumination beam may also be referred to as light beam 20 or simply as beam 20. Furthermore, the illumination of dental surfaces 116 may be in parallel to the line of sight 10 when the angle α is equal to zero. Due to the inhomogeneity of dental surfaces 116, such as, for example, due to the presence of nooks and crannies, it may be difficult to obtain an adequate illumination of the disclosing agent on the dental surfaces 116, which may result in unlit areas, for example, the dental surface 116 in line with the line of sight 10 may not be (adequately) illuminated by the illumination beam 20 due to, for example, casting shadows that limited the illumination of the dental surface 116 of interest. Therefore, in the area where no (adequately) illumination may be supplied the observation of the dental plaque X1 via use of a dental plaque X1 disclosing agent is not feasible as dental plaque X1 disclosing agents need to be photoexcited to produce a fluorescence signal. In order to reveal the dental plaque X1 on arbitrary dental surfaces 116 to the user, the system needs to guarantee the interaction between illumination beam 20, disclosing agent and line of sight 10, regardless of the inhomogeneity of the dental surfaces 116, which may only be achieved if the line of sight 10 is in parallel to the illumination beam 20.

FIG. 3 schematically depicts concepts of illumination of dental surfaces 116 via a kit 100 according to embodiments of the present invention. In simple terms, the observation component 102 may be configured to provide a line of sight 10, which may further be configured to be directed to dental surfaces 116, e.g. a tooth surface. Furthermore, a light source component 104 may be configured to supply a light beam 20, which may further be configured to be directed to dental surfaces 116. In an embodiment of the present invention, the line of sight 10 and the light beam 20 may interact in a common point P, which may, for example, be a point on the dental surface 116 and which may also include a dye component on the point P, i.e. the line of sight 10 and the light beam 20 may converge in a point P and the angle formed between the converging line of sight 10 and light beam 20 may be referred to as illumination angle α, which also be referred to as the interaction angle α or simply as angle α.

It will be understood that whenever a dental surface 116 or a point on a dental surface 116 is mentioned, it is intended to refer to this point P, which may be a point taken as part of the explanation of the geometrical conditions met by embodiments of the present invention.

In simple words, the presence of shadows may be avoided via a steer of the illumination beam in parallel to the line of sight 10 or via minimization of the illumination angle α, which may be achieved via a parallel alignment of the line of sight 10 and the illumination beam 20, which may be also be referred to as small-angle condition. The small-angle condition may be advantageous, as it promotes an undisturbed interaction between the components needed to disclose dental plaque X1.

In one embodiment of the present invention, the angle α may be less than 35 degrees, more preferably less than 25 degrees, most preferably less than 15 degrees. This may be advantageous, as it may allow to solve or least overcome the shortcoming related to insufficiently illuminated dental surfaces 116. Therefore, embodiments of the present invention may derive a model (and its technical implementations) which may parameterize properties of the observation component 102 via a field of view, and may further describe the illumination of the disclosing agent component within the field of view, via a light source component, in order to produce the small-angle condition. Providing illumination, that produces the small-angle condition (and hence enables an interaction between the systems components) for a huge portion of the field of view, may be referred to as point-source illumination.

Furthermore, the interaction between the observation component 102, lights source component and dental disclosing agent may depend on the optical properties of the components, i.e. wavelengths of the illumination beam 20, the absorption properties of the dental disclosing agent and the spectral sensitivity of the observation component. In simple terms, the light source component 104 may be configured to provide light with a wavelength that may be absorbed by a dental disclosing agent; the dental disclosing agent may be configured to absorb the light source component 104, and to emit fluorescent light; the observation component 102 may be configured to collect the fluorescent light of the dental disclosing agent.

For instance, in an embodiment of the present invention, the light source component 104 may be configured to emit light in a wavelength range between 200 and 750 nm, more preferably between 250 and 700 nm, most preferably between 300 and 600 nm.

The dental disclosing agent may be configured to absorb or emit light of wavelengths in a range between 200 and 700 nm, more preferably between 250 and 650 nm, most preferably between 300 and 600 nm.

In one embodiment of the invention, the observation component 102 is complemented by a filtering component wherein the filtering component may be configured to pass on wavelengths to the observer, in a range between 350 and 700 nm, more preferably between 400 and 650 nm, most preferably between 450 and 600 nm.

Furthermore, in one embodiment, the light source component 104 may comprise an optical filter, wherein the optical filter may be configured to filter the light of at least one light source, to provide light of the desired wavelength. In another embodiment, the light source component 104 comprises of an LED which may be configured to emit light of the desired wavelength.

In simple words, the light source component 104 may emit light of certain wavelengths. These embodiments describe alternative light sources to produce a certain wavelength, of the at least one light source. The emitter of light may emit light of a broader spectrum; however, this light may be used to create an alternative light source, producing the same properties.

FIG. 4 schematically depicts concepts of an observation component conceptually identified by reference numeral 102, the field of view parameterized via solid angle conceptually identified by reference numeral Ω, with the apex of the solid angle conceptually identified by reference numeral G.

The observation component 102 may be a surface (represented in a side-view in FIG. 4) configured for collecting light, which may be for example CCD chip. The extend, the observation component 102 collects light from, may be conceptually described via the field of view (FoV). The FoV of the observation component 102 may be modeled as a surface on a geometrical sphere. The size of the sphere is arbitrary, as all directions light comes from may be described via a vector from the point G to any point P on the surface of the sphere. The field of view may be parameterized via solid angle Ω. Furthermore, the field of view may be defined from the apex of the solid angle (point G), which may coincide with the center of the observation component. The extend of the field of view depends on the design of observation component. E.g. a camera with a wide-angle lens has a bigger field of view than a normal camera. A mirror exhibits a field of view corresponding to half a sphere (2π steradian).

The Point P, which may be within the field of view of the observation component 102, may be a point on the dental surface 116 which may also include a dental disclosing agent on the point P. The line of sight 10 signifies the direction of the light originating from Point P, that may be collected by the observation component. Thus, the line of sight 10 may be represented as an arrow from the Point P and the point G.

FIG. 5 schematically depicts concepts of interaction between the light source component 104, the disclosing agent and the observation component 102, the position of a light source 104 within a distance from G conceptually identified by reference numeral d, a radial beam geometry parameterized by a beam angle conceptually identified by reference numeral δ and an illumination beam 20 and concepts of illumination isotropy (or illumination-isotropy condition).

The light source component 104 comprises of a light source within a distance d to the point G and a radial beam geometry parameterized by the beam angle δ. Depending on these parameters, the light source component 104 exhibits an illumination geometry, that determines the illumination of a Point P in field of view of the observation component.

The illumination beam 20 signifies the direction a Point P is illuminated from by the light source component 104. Thus, the illumination beam is represented as a line, connecting the Point P and the light source component 104. The line of sight 10 and the illumination beam 20 may converge in a Point P, forming the illumination angle α.

As demonstrated in FIG. 2, minimizing the illumination angle α is advantageous, as it provides that neither line of sight 10 or illumination beam 20 may be blocked, ensuring the interaction between observation component 102, disclosing agent component (at point P) and light source component 104. In the state of the art, the interaction between the components may be blocked for more hidden tooth surfaces. In simple words, the invention describes a condition the embodiments of the present invention need to fulfill, for a reliable disclosure of dental plaques X1, independently of the accessibility of the dental surface 116 in question, which also be referred to as small-angle condition.

For instance, in an embodiment of the present invention, the interaction between a line of sight 10 of the observation component 102 and a light beam 20 of the light source component 104 at the dental disclosing agent exhibits an angle of illumination a equal to or less than 35 degrees, more preferably less than 25 degrees, most preferably less than 15 degrees.

The illuminated Field of View ΩL signifies the entirety of points P in the field of view interacting with an illumination beam 20, and may also be determined by taking the intersection of the field of view of the observation component 102 and the illumination geometry of the light component. Maximizing ΩL is advantageous, as it minimizes all points P in the field of view which are not illuminated (there is no interaction between observation component 102, disclosing agent component and light source component 104, so Plaque on these Points is not revealed). In the state of the art, only a fraction of Ω is illuminated by the light source component 104, so the disclosure of Plaque depends on the handling of the device, producing unreliable results. In simple words, the invention describes a condition the embodiments of the present invention need to fulfill, for a reliable disclosure of Plaque, independently of the handling of the device.

For instance, in an embodiment of the present invention, the light source component 104 irradiates more than 30%, more preferably more than 50%, most preferably more than 70% of the field of view of the observation component. This approach may be advantageous, as an illuminated FoV of more than 70% may include most points of the dental surface 116, which may represent the points reachable by the line of sight 10.

The interaction between observation component 102 and light source component 104 in a point P on ΩL is also characterized by the angle of illumination a. As a result of the interaction of observation component 102 and light source component 104, each point P in ΩL may exhibit a different illumination angle α. In simple words, the illumination angle of a point P may depend on the direction the point is detected from by the observation component 102 (i.e. it depends on the line of sight 10). This condition may also be referred to as illumination anisotropy.

As it is advantageous to illuminate a point P according to the small-angle condition, preferably the amount of points P interacting with the light source component 104 according to the small-angle condition is to be maximized. This condition may also be referred to as illumination-isotropy condition.

For instance, in one embodiment of the present invention, the light source produces an illumination geometry, thereby irradiating more than 30%, more preferably more than 50%, most preferably more than 70% of the illuminated FoV according to the small angle condition.

FIG. 6 schematically depicts concepts of point-source illumination. FIG. 6 depicts the light source component 104 and observation component 102 interacting with certain given parameters. The observation component 102 exhibits a field of view of half a sphere (2π steradian), the light source is at a zero distance to the center of the observation component (point G). The beam angle exhibits 180 degree, illuminating 100% of the field of view. In this illumination geometry, as all Points P (i.e. points P1 and P2) are illuminated with an illumination angle of zero-degree, illumination-isotropy is provided.

FIG. 6 shows as an example, the conditions required to illuminate all points P on Ω according to the small-angle condition. It exemplifies a geometrical condition which may also be referred to as point-source illumination.

As the observation component 102 exhibits a spherically-symmetric geometry of collecting light with respect to the point G, in order to produce the small-angle condition for all points P on the field of view, the geometry of the light source component 104 (illumination geometry), needs to reflect the geometry of the observation component. Therefore, the illumination geometry of the light source component 104 exhibits the same solid angle and is spherically-symmetric to point G as well. Thus, the illumination geometry is similar to the illumination geometry of a point source of light—the condition may also be referred to as point-source illumination. In simple words, the term point-source illumination describes a condition of geometrical alignment of the light source component 104 to the observation component 102 which embodiments of the present invention may be configured to provide illumination characterized by the small-angle condition for arbitrary points P on ΩL, of more than 30% of the field of view. In more simple words, the term point-source illumination is intended to describe an illumination geometry which satisfies the small-angle condition and the illumination isotropy condition.

For instance, in an embodiment of the present invention, the illumination geometry of a point source of light may be mimicked by static solution embodiments or dynamic solution embodiments.

FIG. 7 schematically depicts concepts of a static solution according to embodiments of the invention with a distance conceptually identified by reference numeral di and a body conceptually identified by the reference numeral 110.

A static solution embodiment comprises of a body 110 embedding the observation component 102 and the light source component 104, providing a defined distance di between the point G and the light source component 104. The illumination geometry of the static solution embodiment comprises of a series of geometrical variables, mentioned in the descriptions of FIG. 6, to produce a point-source illumination.

In simple terms, the light source component 104 may be placed within a distance di from the point G, which may further exhibit a beam angle δ. As result of the separation distance di, the illumination angle α for a point P may break the small-angle condition. Moreover, due to the beam angle, the field of view Ω may be illuminated, according to the isotropy condition. However, the closer the distance di to zero and the bigger the beam angle δ, the more accurately point-source illumination may be produced.

In simple words, a static solution may comprise a kit comprising of an observation component 110 and a light source 104, providing a defined distance di between the point G and the light source 104.

For instance, in an embodiment of the present invention, the distance between a light source 104 and the center of the observation component is less than 10 cm, more preferably less than 6 cm, most preferably less than 4 cm.

FIG. 8 schematically depicts concepts of a dynamic solution according to embodiments of the invention with a secondary device component conceptually identified by reference numeral 40, an observation point conceptually identified by reference numeral O, a distance between the light source component 104 and the observation point O conceptually identified by reference numeral de, and a reflection angle conceptually identified by reference numeral β.

A dynamic solution embodiment comprises of a kit comprising an observation component 102, whereas the observation component 102 is reflecting the illumination beam 20 and line of sight 10, and a secondary device component 40 providing a distance de between an observation Point O and the light source 104 on the secondary device. The illumination geometry of the dynamic solution embodiment comprises of a series of geometrical variables, mentioned in the descriptions of FIG. 8, to produce a point-source illumination.

The observation point O is the closest point of the line of sight with respect to the light source component 104. In simple words, the distance de between a point O and the light source component 104 is the closest distance between the line of sight and the light source component 104. In even more simple words, in most embodiments, the point O is the foot of the perpendicular from the line of sight 10.

In a dynamic solution embodiment, a point P1 in the field of view of the observation component 102 is illuminated via a source of light which is placed on a secondary device component 40. The observation component 102 comprises a reflecting surface, redirecting both line of sight 10 and illumination beam 20.

The figure shows a line of sight 10 emerging from point O, directed to the point P1, representing the fluorescent signal of the point P1 collected by the observation component 102 and reflected via an angle β1 to the observation point O. Tilting the observation component 102 to position 50, the line of sight is reflected at an angle β2 to the point P2. In simple terms, by varying the angle β, any points in the field of view may be pictured by the line of sight 10.

Moreover, the illumination beam 20 emerging from the light source component 104 is reflected via the observation component 102 to the point P1 as well. Tilting the observation component 102 to position 50, the illumination beam 20 is reflected to the point P2. In simple terms, by varying the angle β, any points in the field of view may be illuminated by the illumination beam 20. The illumination angle α depends on de, a distance between O and the light source 104, provided by the secondary device component 40. However, the closer the distance de to zero, the better the small-angle condition may be provided, the more accurately point-source illumination may be produced.

For instance, in an embodiment of the present invention, the light source is placed at a distance to the line of sight of less than 15 cm, more preferably less than 10 cm, most preferably less than 6 cm.

For instance, if the secondary device component provides a distance de of zero, the line of sight and an illumination beam 20 are in parallel, so all points P are illuminated with α equals zero.

FIG. 9 schematically depicts a kit for inspection of oral cavities according to a static solution. In simple terms, the kit 100 may comprise an elongate holding handle conceptually identified by reference numeral 206, which may be configured to allocate at least one observation component 102. This kit may be referred to as a dental device 100. In one embodiment, the observation component 102 may be a mirror.

In one embodiment, the elongated holding handle 206 may be made of corrosion and/or temperature resistance material comprising one of: stainless steel, titanium alloys, zirconium alloys, molybdenum alloys, tantalum alloys, copper alloys such as bronze and/or brass, graphite, aluminosilicates, pure silica, quartz glass, andesite and/or basalt coated materials, polyfluoroethylene resins, polyethylene, polystyrene, etc.

The dental device 100 may further comprise a switch to turn on and off a light source 104, which may be configured to provide a point-source illumination by placing a light source within a distance di to point G (the center of the observation component). The dashed circle with radii dL signifies the possible locations of the light source which can be placed anywhere within the circle in order to provide this condition. As the distance of the light source to the observation component 102 may be essential to produce point-source illumination, the distance dL may represent the maximal distance possible to provide point-source illumination in a static solution, which is preferably smaller than 10 cm, more preferably smaller than 6 cm, most preferably smaller than 4 cm. In more simple words, the distance dL may represent the distance required to obtain illumination angles α smaller than 35 degrees.

In one embodiment, the distance di of a static solution embodiment is provided by placing a curved reflecting surface within the distance dL. This may be achieved via a laser pointer configured to emit a small beam of light on a curved reflecting surface close the observation component. In another embodiment, the light source may be built within the elongated holding handle 206 and the light may be transported close to the observation component 102 via optical fiber.

In simple words, the static solution may require the source of light being within distance di from the point G. These embodiments describe alternative light sources, which are technical implementations of the distance di. The source of light may be placed somewhere outside of the distance dL, however, the light may be used to create an alternative light source at a distance di to G, producing the illumination geometry of a static solution.

Furthermore, the light source may be placed in a plurality of positions with respect to the observation component 102. For instance, FIGS. 10, 11 and 12 schematically depict exemplary embodiments for different positioning of the light source component 104.

In one embodiment, as observed in FIG. 10, the light source component may be a single light source, for instance, a single LED allocated with a small distance to the observation component 102, which may be advantageous, as it may provide a small illumination angle. A huge beam angle δ with may facilitate to supply a huge illuminated field of view.

In another embodiment, as observed in FIG. 11, the light source component 104 may comprise of a plurality of light sources, for instance, multiple LEDs, which surround the observation component 102. As the light sources have a small distance to the observation component 102, a small illumination angle is provided. Furthermore, as the light cones of all LEDs combine, illumination shadows from one LED may be counteracted by another LED.

In a further embodiment, as observed in FIG. 12, the light source may be positioned behind a dichroic filter, so the observation component 102 itself may act as a light source, similar to a flashlight. The dichroic filter transmits the wavelength of the illumination beam 20, but reflects the wavelength of the line of sight 10.

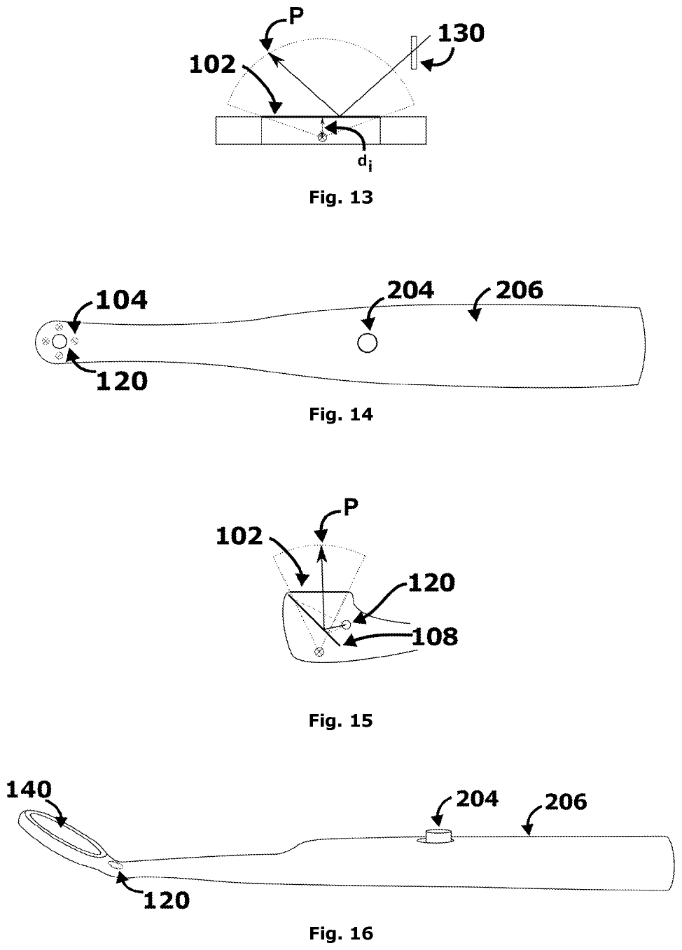

FIG. 13 schematically depicts the positioning of a light source component 104 as shown in FIG. 12 for a dental mirror embodiment as shown in FIG. 9. In simple terms, the observation component 102 is a dichroic filter, transmitting the light of the light source component 104 which is placed behind the observation component 102 within a distance di. However, the observation component 102 reflects the fluorescent signal of a point P to the line of sight. Including a filter 130 within the line of sight 10, the observation component 102 only directs the fluorescent signal to the line of sight to avoid dazzling by the excitation light.

FIG. 14 schematically depicts a dental camera 120 for inspection of dental surfaces 116 according to a static solution. In simple terms, the observation component 102 of the embodiment depicted in FIG. 9 may be a dental camera 120.

In simple terms, the at least one dental camera 120 may be position at the extreme end of the kit 100, and may further comprise at least one light source, which be allocated in a plurality of positions. For instance, FIGS. 10 and 11, schematically depict exemplary embodiments exhibiting different positionings of the light source, but always keeping the distance to the dental camera 120 as small as possible in order to maximize the illumination angle.

FIG. 15 schematically depicts the positioning of a light source component 104 as shown in FIG. 12 for a dental camera 120 embodiment as shown in FIG. 14. In simple terms, the observation component 102 is transmitting both excitation and emission light. The excitation beam of the light source component 104 is transmitted via a dichroic filter 108 through the observation component 102. The observation component 102 collects the fluorescent signal of a point P and transmits it to the dichroic filter 108. The dichroic filter reflects the fluorescent signal to camera 120. As the dichroic filter does not reflect wavelengths of the excitation light, the fluorescent signal is filtered as well, according to a filtering component 130 embodiment.

In a further embodiment, the dental device 100 depicted in FIG. 9 may comprise of multiple observation components. As observed in FIG. 16, it may comprise, for example, a dental mirror 140 and a dental camera 120. The image data captured by the camera 120 may be used to analyze toothbrushing habits, the dental mirror 140 and camera 120 may also include a filtering component.

The present invention may also comprise a filtering component 130 configured to filter wavelengths of the light source, which may further be configured to improve the contrast of the picture. The filtering component may be placed in a plurality of positions. For instance, in one embodiment depicted in FIG. 9, the filtering component may be a (optical) filter, which may be placed on the dental mirror, either within the dental mirror glass, or on the elongated holding handle 206. Furthermore, the filtering component may also be placed on a secondary device 40, e.g. an external mirror, or glasses with optical filters.

In one embodiment, depicted in FIG. 14, the observation component 102 may be a dental camera 120 provided with a light source component 104. The filtering component may be placed on the dental camera 120, which may be an optical filter placed in front of the camera lens and/or a spectral sensitivity of a photosensor. Another embodiment may also comprise a software processing component to filter the light.

FIG. 17 schematically depicts devices for inspection of oral cavities according to a dynamic solution embodiment. It may comprise of two devices, for instance, a dental device 100 comprising of an observation component 102 capable of reflecting the illumination beam 20 and line of sight 10, and a secondary device 40 comprising a light source component 104, providing a distance de between an observation Point O and the light source component 104 embedded in the secondary device 40. The secondary device may also comprise a filtering 130 component.

In simple terms, the dental device 100 of a dynamic solution embodiment may comprise of a dental device 100 of a static solution, which comprises a reflecting observation component 102, and which may not comprise the light source component 104.

In simple terms, the secondary device of a dynamic solution may comprise of the light source component 104, designed to produce point source illumination of dental surfaces 116, which may be used in combination with a dental device 100.

As the distance of the light source to the line of sight may be essential to produce point-source illumination, the distance de is preferably smaller than 15 cm, more preferably smaller than 10 cm, most preferably smaller than 6 cm. In more simple words, the distance de may represent the distance required to obtain point source illumination.

In one embodiment the body of the secondary device 40 may be made of corrosion and/or temperature resistance material comprising one of: stainless steel, titanium alloys, zirconium alloys, molybdenum alloys, tantalum alloys, copper alloys such as bronze and/or brass, graphite, aluminosilicates, pure silica, quartz glass, andesite and/or basalt coated materials, polyfluoroethylene resins, polyethylene, polystyrene, etc.

The secondary device may further comprise a switch to turn on and off a light source, which may be configured to provide a point-source illumination by placing a light source within a distance de to point O.

In one embodiment the dynamic solution a secondary device 40 may comprise a light source component 104, positioned to provide an illumination beam 20 at a distance de to a glass mirror 150, as schematically depicted in FIG. 18.

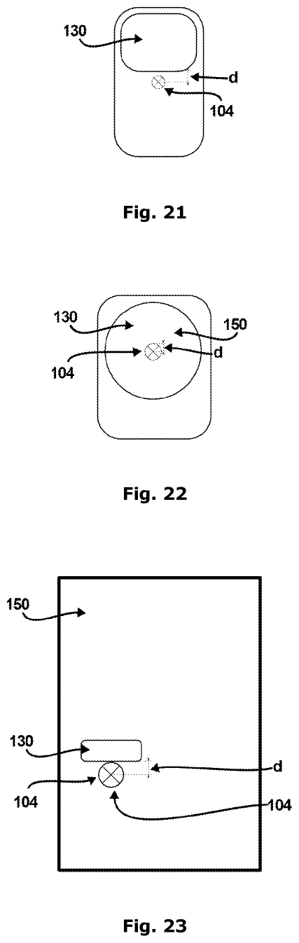

Furthermore, the secondary device may also comprise a mirror such as FIG. 21, or a parabolic mirror in order to magnify the picture of the mouth and a source of light in the center of the parabolic mirror, such as FIG. 22. The filtering component 130 may be incorporated in the mirror glass 150.

The secondary device may also comprise a mechanism to fix the secondary device on a smooth surface, which may be a mirror surface, such as in FIG. 23.

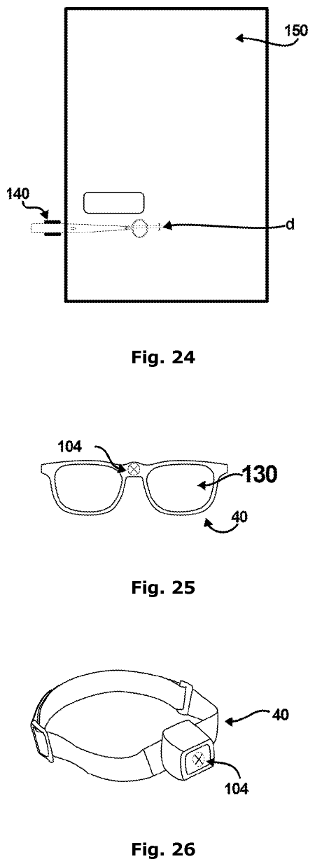

It may also comprise of a second dental device 100 which comprises a source of light, and may be fixed in front of a mirror surface 150 such as in FIG. 24. The secondary device may be expanded by a filtering component 130 may be fixed on the mirror surface 150.

In simple terms, one embodiment may comprise a dental device 100, such as, for example, a dental mirror, and a secondary device, such as, for example, an external mirror. The illumination angle α may depend on the separation distance (between the light source of the secondary device and the closest point of the line of sight) conceptually identified by reference numeral de. For these embodiments, the separation distance de may be the closest distance between the light source and a point of a mirror's surface 150 (as the line of sight is directed via the mirror). The dynamic solution may, in simple terms, exhibit parameters such as, for example, angle of illumination a lower than 35 degrees and a distance de between the source of light to a mirror's surface lower than 6 cm.

In one embodiment of the dynamic solution, a secondary device 40 may comprise of a light source component 104 positioned to provide an illumination beam 20 at a distance de to the line of sight, as schematically depicted in FIG. 19.

Furthermore, the secondary device may also comprise a source of light placed on glasses, such as in FIG. 25, or it may comprise of a source of light on a headband, such as FIG. 26. The source of light may be electronically directed to the mouth, so that the line of sight and illumination beam 20 are in near-parallel independently of head-movements. The secondary device may be expanded by a filtering component 130 which may be incorporated in the glasses such as in FIG. 25.

In simple terms, the embodiment may comprise of a dental device 100, such as, for example, a dental mirror, and a secondary device, such as, for example, a headband or glasses. In such an embodiment, the illumination angle α may depend on the separation distance de between the source of light and the line of sight, which may be approximately the distance between the source of light and the position of the eyes.

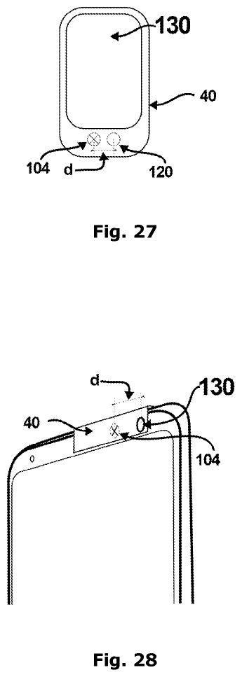

In one embodiment of the dynamic solution, a secondary device 40 may comprise a light source component 104 positioned to provide an illumination beam 20 at a distance de to a camera 120, which may be directed in the oral cavity, as schematically depicted in FIG. 20. Furthermore, the secondary device may also comprise of a camera and/or a display to display a camera image, such as FIG. 27. It may comprise a filtering component 130 which may be an optical filter in front of the camera, filtering software, or a spectral sensitivity of the photosensor.

The secondary device 40 may also comprise a source of light which can be fixed on a camera device 120, i.e. on a smartphone, such as FIG. 28. It may comprise a filtering component 130 which may be an optical filter in front of the camera or filtering software.

In simple terms, the embodiment may comprise of a kit 100, such as, for example, a dental mirror or a dental camera 120, and a secondary device 40, such as, for example, a light source 104 which may be placed in the proximity of a camera 120. In such an embodiment, the illumination angle α may depend on the separation distances de between the source of light 104 and the position of the camera 120. In simple words, most significant for the illumination angle α may the distance between be the camera 120 and a light source 104 on a secondary device 40, which may simple be referred to as distance de, and which may be lower than 6 cm.

While in the above, a preferred embodiment has been described with reference to the accompanying drawings, the skilled person will understand that this embodiment was provided for illustrative purpose only and should by no means be construed to limit the scope of the present invention, which is defined by the claims.

Whenever a relative term, such as “about”, “substantially” or “approximately” is used in this specification, such a term should also be construed to also include the exact term. That is, e.g., “substantially straight” should be construed to also include “(exactly) straight”.

Whenever steps were recited in the above or also in the appended claims, it should be noted that the order in which the steps are recited in this text may be accidental. That is, unless otherwise specified or unless clear to the skilled person, the order in which steps are recited may be accidental. That is, when the present document states, e.g., that a method comprises steps (A) and (B), this does not necessarily mean that step (A) precedes step (B), but it is also possible that step (A) is performed (at least partly) simultaneously with step (B) or that step (B) precedes step (A). Furthermore, when a step (X) is said to precede another step (Z), this does not imply that there is no step between steps (X) and (Z). That is, step (X) preceding step (Z) encompasses the situation that step (X) is performed directly before step (Z), but also the situation that (X) is performed before one or more steps (Y1), . . . , followed by step (Z). Corresponding considerations apply when terms like “after” or “before” are used.

1. A kit configured to perform oral cavity inspection comprising at least one of
an observation component;
a light source component; and
a dental disclosing agent applied on dental surfaces,
wherein interaction between a line of sight of the observation component and a light beam of the light source component at the dental disclosing agent exhibits an angle of illumination (α) equal to or less than 35 degrees.

2. The kit according to claim 1, wherein the angle of illumination (α) is less than 25 degrees, more preferably less than 15 degrees.

3. The kit according to claim 2, wherein the kit further comprises a filtering component wherein the filtering component further comprises at least one of
an optical filter;
a spectral sensitive sensor; and
a filtering software,
wherein the filtering component is configured for passing the fluorescent emission of the dental disclosing agent and blocking light of wavelengths via selectively filtering light in a wavelength range between 350 and 700 nm, more preferably between 400 and 650 nm, most preferably between 450 and 600 nm.

4. The kit according to claim 1, wherein the kit comprises an energy source component comprising at least one energy source comprising at least one of
a battery;
a rechargeable battery and/or accumulator;
a replaceable battery and/or replaceable accumulator;
a transformer;
a capacitor; and
wherein the energy source component comprises at least one electronic control mechanism configured to switch on and off the at least one energy source.

5. The kit according to claim 1, wherein the dental disclosing agent comprises at least one dye of, for example, but not limited to, iodiones such as iodine crystals, potassium iodide, zinc iodide, azo dyes such as bismarck brown Y, merbromin, erythrosine, fast green, fluorescein, fuchsin, riboflavin, etc., and their derivatives.

6. The kit according to claim 1, wherein the light source component comprises at least one light source configured to emit light with wavelengths between 200 and 750 nm, more preferably between 250 and 700 nm, most preferably between 300 and 600 nm.

7. The kit according to claim 1, wherein the observation component is further configured to collect light from a field of view higher than 1 steradian, more preferably higher than 2 steradian, most preferably higher than 3 steradian.

8. The kit according to claim 1, wherein the observation component is configured to
capture fluorescence signals from dental surfaces via at least one observation component; and/or
reflect the captured fluorescence signal from dental surfaces; and
wherein the observation component further comprises at least one of
a dental mirror; and
a dental camera.

9. The kit according to claim 1, wherein the observation component comprises an elongated holding handle made of corrosion and/or temperature resistance material comprising one of: stainless steel, titanium alloys, zirconium alloys, molybdenum alloys, tantalum alloys, copper alloys such as bronze and/or brass, graphite, aluminosilicates, pure silica, quartz glass, andesite and/or basalt coated materials, polyfluoroethylene resins, polyethylene, polystyrene, etc.

10. The kit according to claim 1, wherein the light source component is placed at a distance of less than 10 cm from the center of the observation component, more preferably less than 6 cm, most preferably less than 4 cm from the light source component.

11. The kit according to claim 10, wherein the light source component comprises of at least one light source which is further configured to provide a beam angle larger than 30 degrees, more preferably larger than 60 degrees, most preferably larger than 90 degrees.

12. The kit according to claim 1, further comprising a secondary device component comprising at least one of
a second observation component;
a filtering component;
a light source component; and
an energy source component;
wherein the second observation component is at least one of
a remote observation device;
an external camera; and
an external mirror.

13. The kit according to claim 12, wherein the light source component of the secondary device component is placed at a distance of less than 15 cm to the line of sight, more preferably less than 10 cm, most preferably less than 6 cm.

14. The kit according to claim 1, wherein the kit is configured to illuminate more than 30%, more preferably more than 50%, most preferably more than 70% of the field of view of the observation component with an angle of illumination (α) equal to or less than 35 degrees, more preferably less than 25 degrees, most preferably less than 15 degrees.

15. The kit according to claim 1, wherein the kit is configured to achieve a Michelson contrast between the dental plaque disclosing agent on dental surfaces and the clean dental surface higher than 0.03, more preferably higher than 0.04, most preferably higher than 0.05.

16. The kit according to claim 3, wherein the kit comprises an energy source component comprising at least one energy source comprising at least one of
a battery;
a rechargeable battery and/or accumulator;
a replaceable battery and/or replaceable accumulator;
a transformer;
a capacitor; and
wherein the energy source component comprises at least one electronic control mechanism configured to switch on and off the at least one energy source.
